(19) United States
(12) Patent Application Publication
(10) Pub. No.: US 2019/0282243 A1
(43) Pub. Date: Sep. 19, 2019

(54) Location Control Apparatus for Bone Resection

(71) Applicant: A Plus Biotechnology Company Limited, New Taipei (TW)

(72) Inventors: Kai-Hsing Wu, Taipei (TW); Hsiang-Wei Lo, New Taipei (TW); Kun-Jhih Lin, Taichung (TW); Ping-Sheng Yu, Taipei (TW)

(73) Assignee: A Plus Biotechnology Company Limited, New Taipei (TW)

(21) Appl. No.: 16/005,764

(22) Filed: Jun. 12, 2018

(30) Foreign Application Priority Data
Mar. 19, 2018 (TW) 107109296

(51) Int. Cl. A61B 17/15; A61B 17/17; A61B 90/00

(52) U.S. Cl. CPC A61B 17/151; A61B 2090/067; A61B 90/06; A61B 17/1764

(57) ABSTRACT

The present invention is a location control apparatus for bone resection, the location control apparatus includes a guiding module, located at the bottom of the location control apparatus for bone resection, a blocking plate module, formed on the rear end of the guiding module and has a protruding shape, and, a guide groove, located at the bottom of the blocking plate module to allow a osteotome or bone saw to be inserted, wherein the dimension of the inserted osteotome or bone saw could be adjusted.

### BACKGROUND OF THE INVENTION

### Technical Field

The present invention relates to a location control apparatus for bone resection to prevent over-cutting during operation, more particularly, the present invention relates to a location control apparatus fixed on a bony resection apparatus for controlling the accessing depth to limit the osteotome or bone saw to over-reach a default depth, to avoid the injuring of the bone or surrounding soft tissues.

### Description of Related Art

Bone resection is a method of orthopedic treatment such as osteotomy. Osteotomy aims to remove a part of the bone around the joint, making rearrangement of the limb alignment, and fixation for achieving the purpose of correcting the bone structure or replacing the artificial articulation under the situation of bone damage, for instance, the kyphosis, resulting from poor posture, or congenital neural and muscular abnormalities; the patient with inguinal area pain and activity constraint before middle age, causing by hip dysplasia; walking or movement function degradation resulting from degenerative arthritis with the worn knee articulation and so on. In order to solve the above problems, osteotomy may be used as a treatment to reduce pain, thereby improving walk ability or movement function, delaying articular cartilage degeneration, delaying or avoiding joint replacement surgery. In general, osteotomy is usually performed at the early stage of bone pathogenesis. The outcome of severe bone pathogenesis usually is inferior.

Taking the High Tibia Osteotomy (HTO) as an example, the HTO is mainly used to treat the younger patients suffering from degenerative arthritis of the medial tibial compartment but still with good functional activities (the age of less than 55 years old). Generally, the degenerative arthritis is caused by wear of medial tibia articulation, it results in more stress on the medial articular cartilage and collapse of medial tibial plateau leading to an “O” shaped leg.

The principle of high tibia osteotomy is to use a closed or open wedge-shaped bony displacement at the metaphysis 103 to correct the knee joint alignment, making the mechanical axis could transfer from medial tibia articulation to intermediate or lateral tibia articulation for relieving the stress on the medial tibia cartilage. After the osteotomy surgery, the closed or open wedge is then maintained by the in-vivo or in-vitro fixator to maintain bone structure stability, making the mechanical axis could be restored to normal, so that degenerative arthritis in tibia could be relieved. If the degeneration of the joint progresses in the future, it would be much easier to have artificial joint replacement.

Please refer to FIG. 1A and FIG. 1B, the steps of the high tibia osteotomy are shown as follows. First, a bone resection line 101 cross over the tibia is made on the proximal tibia. Second, according to the deformity angle of the mechanical axis, a wedge is opened to a predetermined angle α, at this step, the low limb alignment would more valgus in appearance than pre-operative, thus, the bearing load of the lateral cartilage would increase and that of the medial cartilage would decrease. Finally, the bone grafting (autogenous, allograft, or artificial bone) will be used to support the open wedge and fixed with the bone plate and the bone screw after the high tibia osteotomy.

As mentioned above, in general, a bone saw or osteotome is used for osteotomy surgery. Ideally, although the bone shape of the patient would be scanned via X-ray before the osteotomy surgery for assisting the surgeon to plan the desired correction angle, depth of bone resection line 101, and wedge of the predetermined angle α. However, since it is not possible monitoring the bones of patients with X-ray continuously, furthermore, the patient's blood would block the scale on the bone saw or osteotome since bleeding during the osteotomy surgery, and the bone strength of is usually very strong, resulting in the surgeon hardly to control the force applied to the bone saw or osteotome, thus, it often makes the bone saw or osteotome over-cut of the tibia, that is, the bone saw or osteotome cutting is too deep to have unexpected injury to the surrounding soft tissue, such as nerves, muscles, and blood vessels resulting in the longer recovery after osteotomy surgery.

In the prior art, such as Taiwan Patent No. 1361677 and US Patent No. 20100036404 A1, the two citations both disclosed a safety scalpel that can adjust the length of a surgical blade by a blade and a slot fixed on the bottom of the retainer. The length of the scalpel could be fixed and slid back and forth, so that the surgical blade could adjust the required length when cutting. However, as mentioned before, since the structural strength of the bone is usually very strong, cutting the bone with a scalpel could not meet the actual needs of the osteotomy, so it is still necessary to have a safety apparatus for the osteotomy operation which could ensure that the desired depth of the bone resection line 101 is correct, and the surgical risk caused by over-cut will not occur due to improper control by the surgeon.

### SUMMARY OF THE INVENTION

Accordingly, the purpose of the present invention is to provide a location control apparatus for osteotomy surgery to constrain the cutting depth to prevent the soft tissue and bone from being over-cutting when the osteotomy surgery is performed.

Another purpose of the present invention is to provide a location control apparatus for bone resection, it includes a guiding module to carry the other elements of the location control apparatus thereon; a blocking plate module is formed on the rear end of the guiding module and the blocking plate module has a protruding shape; a guide groove is located at the bottom of the blocking plate module to allow the osteotome or bone saw to be inserted in the direction perpendicular to the plane of the guiding module; wherein the location of the inserted osteotome or bone saw could be adjusted according to the bone resection line.

According to one aspect of the present invention, the scale is disposed on the bone saw, and the bone saw could be slid back and forth to adjust the location after the bone saw insertion into guide groove. When the bone resection is performed, once the bone saw is cutting into a predetermined depth, the blocking plate module would be stuck on the rest portion of the bone to stop the bone saw cutting into deeper portion of the bone continuously to achieve the purpose of the present invention for preventing soft tissue and bone from unexpected injury caused by the over-cutting.

According to one aspect of the present invention, the number of guide groove located at the bottom of the blocking plate module may be plurality, allowing the surgeon to adjust the location of bone saw according to the requirement of the bone resection, which may raise the versatility of the location control apparatus.

According to one aspect of the present invention, wherein the length L of the location control apparatus could be formed based on the need of the bone resection, in another aspect of the present invention, it could be but not limited with 25-100 mm.

According to one aspect of the present invention, wherein the material of the location control apparatus may be rubber, metal (e.g. Titanium alloy or Ti₆Al₄V), composite material or wood with well biocompatibility to prevent allergy during the bone resection process while the location control apparatus touch to surgical location.

According to one aspect of the present invention, wherein the blocking plate module is disposed at least one screw hole for fixing the location control apparatus on the bone saw for preventing the bone saw from being slip off caused by applying too much force on the bone saw.

In one embodiment of the present invention, wherein the bone saw and osteotome is disposed plural location-constrain hole, making the screw to connect with bone saw or osteotome through the screw hole on bone resection apparatus, fixing the bone resection apparatus on the bone saw or osteotome to reach the purpose of location constrain in the present invention.

According to one aspect of the present invention, wherein the bone saw and osteotome further comprises a location-constrain slot in the form of a long strip so that the screw could be locked on the location-constrain slot, fixing the bone resection apparatus on the bone saw or osteotome to reach the purpose of location constrain in the present invention.

### BRIEF DESCRIPTION OF THE DRAWINGS

The components, characteristics and advantages of the present invention may be understood by the detailed descriptions of the preferred embodiments outlined in the specification and the drawings attached:

FIG. 1A illustrates the predetermined location of bone resection line which processed via the osteotemy surgery.

FIG. 1B illustrates the predetermined angle α of bone structure correcting which processed via the osteotemy surgery.

FIG. 2 illustrates the location control apparatus structure of the present invention.

FIG. 3 illustrates an embodiment wherein the location control apparatus is fixed on the bone saw.

FIG. 4A illustrates a font perspective view of a location control apparatus according to the present invention.

FIG. 4B illustrates a font perspective view of another size of location control apparatus according to the present invention.

FIG. 5A illustrates one embodiment wherein the location control apparatus is applied to osteotome.

FIG. 5B illustrates another embodiment wherein the location control apparatus is applied to osteotome.

FIG. 5C illustrates the other embodiment wherein the location control apparatus is applied to osteotome.

FIG. 6A illustrates the location control apparatus is applied to osteotome according to another aspect of the present invention.

FIG. 6B illustrates the location control apparatus is applied to osteotome according to another aspect of the present invention.

### DETAILED DESCRIPTION

Some preferred embodiments of the present invention will now be described in greater detail. However, it should be recognized that the preferred embodiments of the present invention are provided for illustration rather than constraining the present invention. In addition, the present invention can be practiced in a wide range of other embodiments besides those explicitly described, and the scope of the present invention is not expressly constrained except as specified in the accompanying claims.

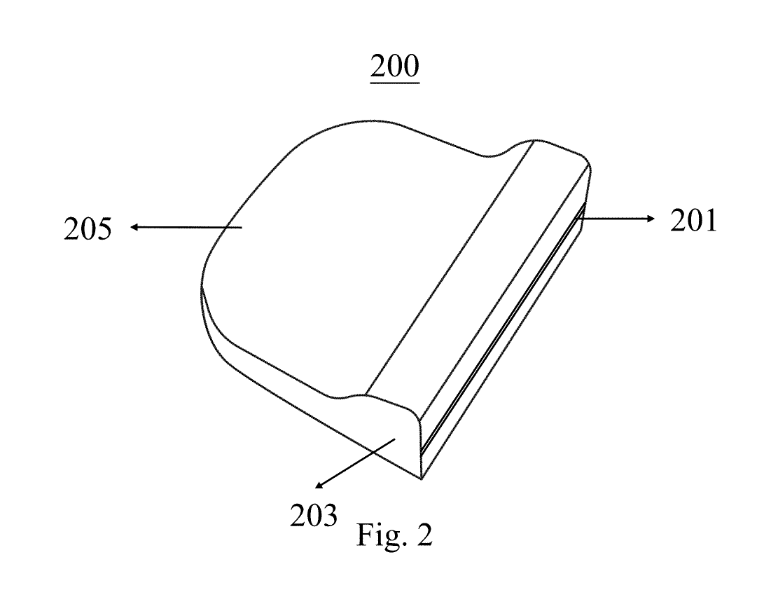

For the purpose to improve the disadvantage of the prior art, please refer to FIG. 2, the present invention is providing a location control apparatus 200 for bone resection, location control apparatus 200 includes a guiding module 205 to carry the other elements of the location control apparatus 200. A blocking plate module 203 is formed on the rear end of the guiding module 205 and has a protruding shape; and, a guide groove 201 is located at the bottom of the blocking plate module 203 to allow osteotome 503 or bone saw 300 to be inserted in the direction perpendicular to the plane of said guiding module 205; wherein the location of inserted osteotome 503 or bone saw 300 could be adjusted according to the bone resection line 101.

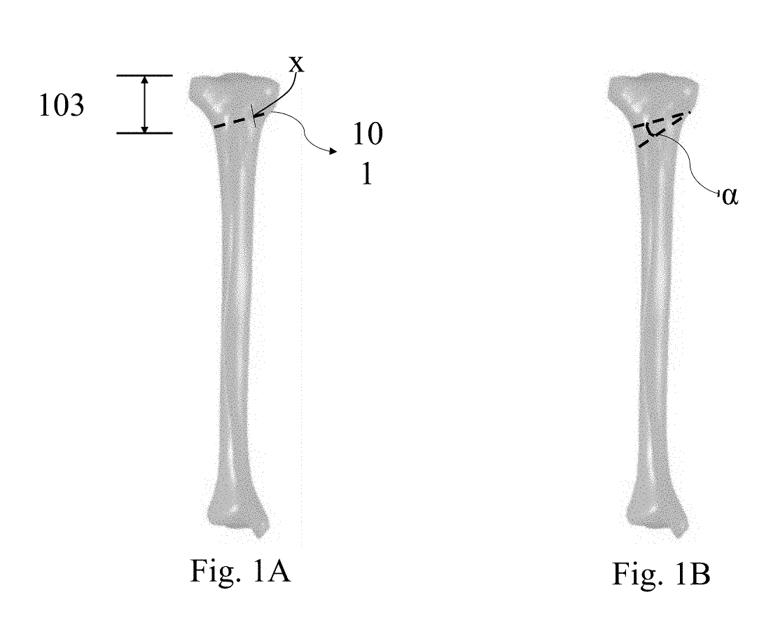

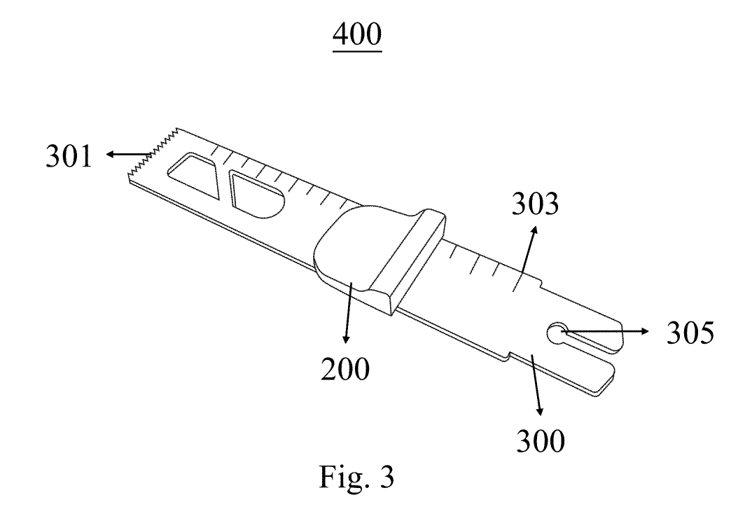

According to one embodiment of the present invention, please refer to FIG. 3, the bone saw apparatus 400 is combined after the bone saw is inserted into the guide groove 201, wherein scale 303 is marked on the bone saw 300, the location control apparatus 200 could be slid back and forth to adjust the location and fixed on the bone saw 300 by the elasticity of the location control apparatus 200. Please refer to FIG. 1A, the bones shape of the patient is scanned via X-ray before the osteotemy surgery is executed, the surgeon then plan the desired depth due to the scanning result of X-ray, indicating that bone saw 300 should cut to x-spot (the x-spot is at the bone resection line 101), thus the location control apparatus 200 is fixed at the scale 303 respect to the location of x-spot, and the bone saw apparatus 400 is fixed to a holder by a clamp module 305 for the operation of bone saw apparatus 400.

As mentioned above, when the surgery starts, the surgeon cuts to predetermined x-spot by the saw tooth portion 301 in front of bone saw apparatus 400, then the blocking plate module 203 would be stuck to the rest portion of the bone to stop bone saw apparatus 400 being moved into the deeper portion of the bone continuously to achieve the purpose of the present invention for preventing the soft tissue and bone from being unexpected injury which is caused by over-cutting. The characteristic of the present invention is to avoid the scale 303 on the bone saw 300 will not be blocked by the blood of the patient during the bone resection. In the meantime, surgeon could apply force harder without worrying the bone over-cut issue which will cause unexpected injury to the rest portion of bone and soft tissue.

According to one embodiment of the present invention, wherein the number of guiding module 201 located at the bottom of the blocking plate module 203 may be plural, allowing the surgeon to adjust the location of bone saw 300 according to the requirement of the bone resection, therefore, the present invention may improve the versatility of bone resection.

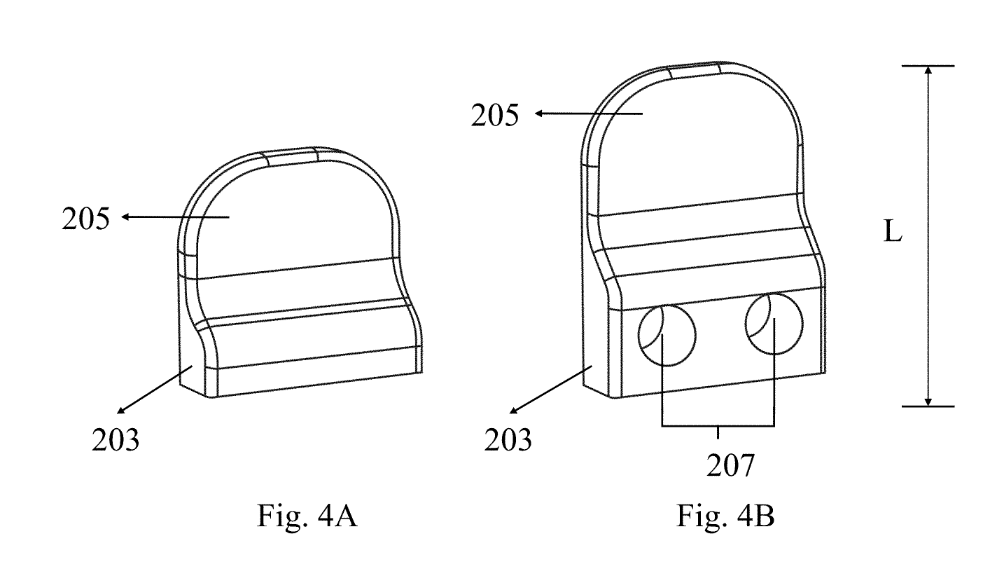

According to one embodiment of the present invention, please refer to FIG. 4A and FIG. 4B, wherein the length L of the location control apparatus 200 could be formed based on the need of the bone resection, in one aspect of the present invention may be 25-100 mm but not limit to above size and the present invention could apply to the bone resection of tibia, sacrum, femur, sacrum, ulna, tibia, clavicle, scapula.

According to one embodiment of the present invention, wherein the material of the location control apparatus 200 may be rubber, metal (e.g. Titanium alloy or Ti₆Al₄V), composite material or wood with well biocompatibility to prevent allergy during the bone resection process while the location control apparatus 200 touch to surgical location.

According to one aspect of the present invention, wherein the blocking plate module 203 is disposed at least one screw hole 207 for fixing the location control apparatus 200 on the bone saw 300 for preventing unexpected risk cause by applying too much force on bone saw 300.

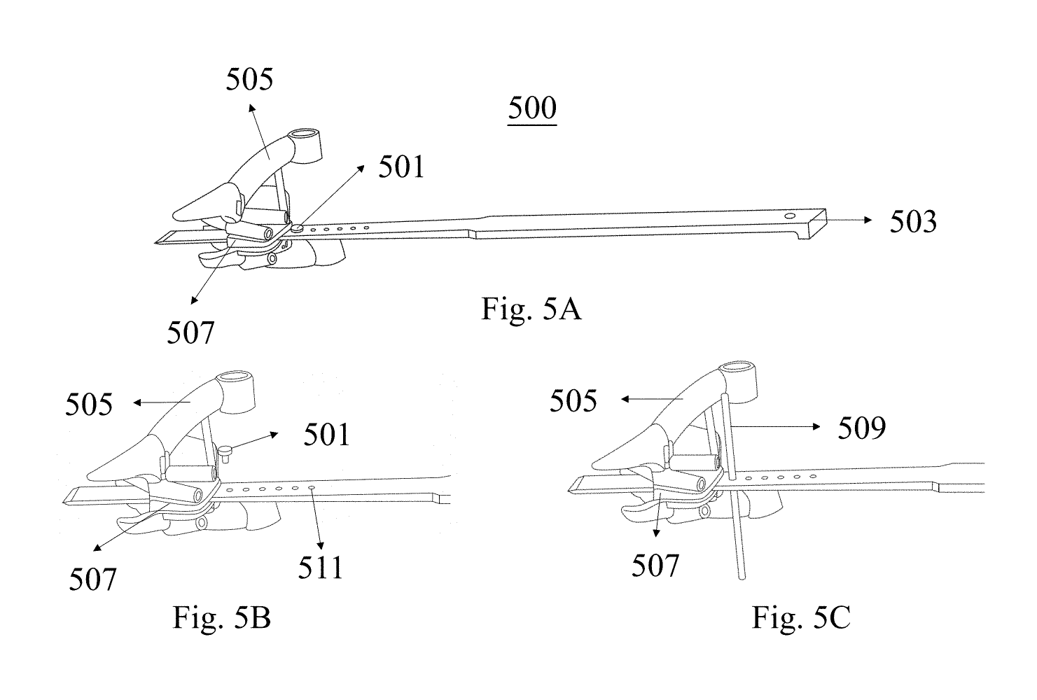

According to one embodiment of the present invention, please refer to FIG. 5A, FIG. 5B, and FIG. 5C, the location control apparatus 507 may also apply to osteotome 503. The figure mentioned above illustrate a osteotome apparatus 500 which includes an bone resection apparatus 505 fixed the osteotome apparatus 500 on bone when the bone resection is operated. A location control apparatus 507 is disposed on the osteotome apparatus 505. A osteotome 503 is disposed plural location-constrain hole 511 for fixing the bone resection apparatus 505 on the osteotome 503 by screw 501.

According to one embodiment of the present invention, the location control apparatus 507 could be fixed on the bone saw 503 by the elasticity of the bone resection apparatus 505.

As mentioned earlier, the surgeon could adjust the location of the osteotome 503 via sliding back and forth, please refer to FIG. 5B, according to the disclose of the present invention, the osteotome 503 is disposed with plural location-constrain hole 511, making the screw 501 connect with osteotome 503 through the screw hole 207 on location control apparatus 507 for fixing the location control apparatus 507 on the osteotome 503 to reach the purpose of location constrain.

As mentioned as the embodiment above, said bone resection apparatus 505 could apply to the bone resection of tibia, sacrum, femur, sacrum, ulna, tibia, clavicle, scapula according to the actual need.

The embodiment as mentioned above may apply to high tibia osteotomy: first, after bone resection apparatus 505 is disposed on the surface of tibia, the surgeon plans the desired depth after the tibia shape scanning result of patient is obtained by X-ray, the following step is to confirm whether the disposition of bone resection apparatus 505 on tibia correct or not. Next, the surgeon locks the screw 501 into location-constrain hole 511 which respect to the predetermined depth. Finally, the bone is cut by the osteotome 503 along to the bone resection line 101 until the predetermined depth of the bone is reached, then the screw 501 would be stuck the bone resection apparatus 507 to stop osteotome 503 cutting into deeper portion inside the tibia to reach the purpose of the present invention for preventing over-cut issue.

According to another embodiment of the present invention, please refer to FIG. 5C, the screw 501 may be replaced by a location constrain element 509 with long-strip form, the disposition of the location constrain element 509 not only could constrain the cutting depth, but providing a visually reference line as well, allowing the surgeon to determine whether the cutting angle and depth of bone resection is correct or not via the relative location between the visually reference line and the tibia for ensuring the accuracy of the predetermined plan.

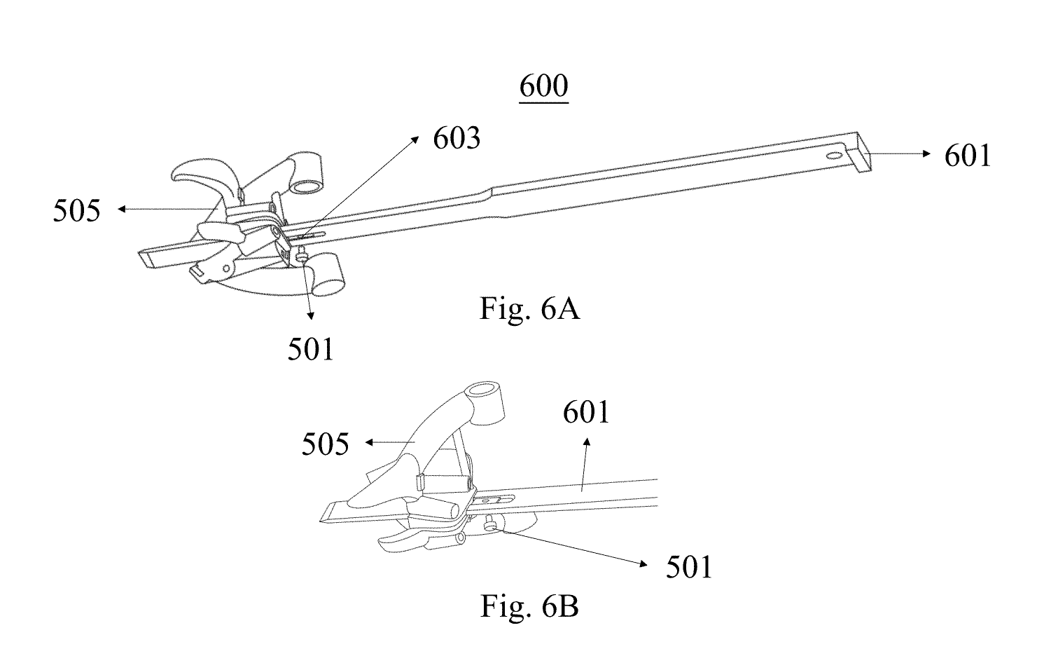

According to the other embodiment of the present invention, please refer to FIG. 6A and FIG. 6B. In the osteotome apparatus 600, a location constrain slot 603 having a long strip shape is disposed on the osteotome 601, wherein the location constrain slot 603 is located at the central axis of osteotome 601, and its width is matched the screw 501, so that the screw 501 could be inserted into the location-constrain slot 603 through the location control apparatus 507. To adjust the location of osteotome 601, the screw 501 could be slid back and forth on the location constrain slot 603, then locking the screw 501 on the location-constrain slot 603, fixing the bone resection apparatus 507 on osteotome 601 to reach the purpose of location constrain in the present invention.

As will be understood by persons skilled in the art, the foregoing preferred embodiment of the present invention illustrates the present invention rather than constraining the present invention. Having described the invention in connection with a preferred embodiment, modifications will be suggested to those skilled in the art. Thus, the invention is not to be constrained to this embodiment, but rather the invention is intended to cover various modifications and similar arrangements included within the spirit and scope of the appended claims, the scope of which should be accorded the broadest interpretation, thereby encompassing all such modifications and similar structures. While the preferred embodiment of the invention has been illustrated and described, it will be appreciated that various changes can be made without departing from the spirit and scope of the invention.

What is claimed is:

1. A location control apparatus for bone resection, comprising:
a guiding module located at a bottom of the constrain apparatus;
a blocking plate module formed on a rear end of the guiding module, wherein said blocking plate module has a protruding shape; and,
a guide groove located at a bottom of the blocking plate module to insert a osteotome or bone saw;
wherein said osteotome or bone saw could be adjusted after insertion.

2. The location control apparatus for bone resection of claim 1, wherein at least one screw hole is disposed at the blocking plate module for fixing the location control apparatus.

3. The location control apparatus for bone resection of claim 1, wherein a length of the location control apparatus is 25-100 mm.

4. The location control apparatus for bone resection of claim 1, wherein a material of the location control apparatus is rubber, metal, composite material or wood.

5. A osteotome apparatus, comprising:
an bone resection apparatus to fixed the osteotome apparatus on a bone when an bone resection is operated;
a location control apparatus disposed on the osteotome apparatus; and,
a osteotome slid back and forth on the location control apparatus to adjust the location and fixed the location on the osteotome apparatus by the elasticity of the location control apparatus.

6. The osteotome apparatus of claim 5, wherein the osteotome further comprises plural location-constrain hole with at least one screw to fix the osteotome on the bone resection apparatus.

7. The osteotome apparatus of claim 5, wherein the osteotome further comprises a location-constrain slot in the form of a long strip so that the at least one screw is locked to constrain a location of the osteotome.

8. The osteotome apparatus of claim 5, wherein a length of the location control apparatus is 25-100 mm.

9. The osteotome apparatus of claim 5, wherein a material of the location control apparatus is rubber, metal, composite material or wood.
